(19) United States
(12) Patent Application Publication
(10) Pub. No.: US 2008/0107689 A1
(43) Pub. Date: May 8, 2008

(54) Stable Microbial Inoculants and Methods for Production of Them

(76) Inventor: Pekka Seiskari, Luoma (FI)

Correspondence Address: CANTOR COLBURN, LLP, 20 Church Street, 22nd Floor, Hartford, CT, 06103, US

(21) Appl. No.: 11/794,348

(22) PCT Filed: Dec. 30, 2005
(86) PCT No.: PCT/FI05/00559

§ 371 Date: Jun. 27, 2007

(30) Foreign Application Priority Data
Dec. 31, 2004 (FI) 20041704

(51) Int. Cl. A61K 39/00; A61K 39/002; A61K 39/02; C12N 1/00; C12N 1/10; C12N 1/14; C12N 1/16; C12N 1/20; A61P 41/00

(52) U.S. Cl. 424/234.1; 424/184.1; 424/274.1; 435/243; 435/254.1; 435/255.1; 435/252.1; 435/258.1; 435/253.5

(57) ABSTRACT

The invention relates to an inoculant in the form of a stable storage paste including a microorganism, a solid carrier, one or more protective substances, and water. The invention further relates to a method for producing of the inoculant using a growth medium including a solid carrier.

### FIELD OF THE INVENTION

The present invention relates to stable, water containing microbial inoculants and to methods for production of water containing microbial inoculants in paste form having excellent storage stability.

The function of inoculants is based on the activity of living microorganisms. Such products comprise biological control agents, mycorrhizal inoculants, inoculants of nitrogen fixing bacteria, probiotics, bakers yeast, spawn of edible mushrooms and lactic acid bacteria for silage preservation.

### BACKGROUND OF THE INVENTION

Good storage stability is essential to microbial inoculants. The shelf life of such products for example for agricultural applications should be at least 3 months, preferably 12 months.

Microbial inoculants are usually stabilized by drying, which is a good method to achieve long shelf life for spore forming microbes. However, many microbes and nematodes do not form durable spores and therefore their drying can be complicated and very expensive or even impossible. Drying of living microbes is a very demanding unit operation and usually some viability is always lost depending on the drying method. Drying is also very vulnerable to contaminations in processes where strict asepsis is required.

Living microbes can also be preserved in non-dried form by adding some protective agents which stabilize the cell membranes, cease the metabolism, adjust the osmotic pressure or act as cryoprotectants. Microbial strains in culture collections are commonly stored in glycerol solutions at very low temperatures. Such methods are not feasible in commercial applications of inoculants.

When microbial inoculants are produced on commercial scale the formulations have to be inexpensive and easy to apply by the end users. Biological control agents, for example, are usually applied as water suspensions by spraying, through irrigation systems, mixed with soil or the plants roots are dipped into the suspension. Also seed dressing or coating is common.

The most common commercial method for the cultivation of any microbe is submerged liquid fermentation. Microbial inoculants are produced by separation of the cell mass and submerged spores from the cultivation broth. However, submerged fermentations have certain generally known drawbacks. Because cells have to be separated from the culture broth substantial amounts of waste liquid is always produced. Further, growth morphology of the microorganisms in liquid cultures does not necessarily favor the formation of durable living units, i.e. spores, which would be ideal for stable products.

An alternative to submerged fermentation is solid state fermentation (SSF). It is well known to a person skilled in the art as a method for cultivating microbes on media where water is impregnated to a solid carrier. The amount of free water is very small contrary to submerged liquid fermentation and the growth morphology of the microbes on the surfaces of solid particles is different from submerged growth.

A few aqueous microbial inoculants have been introduced to the market. Microbial inoculants are usually stored in dry or semi-dry form and applied in a liquid form.

Torres et al. (J. Appl. Microbiol. 94 (330-339) 2003) made a liquid formulation of biocontrol yeast Candida sake. Glycerol or polyethylene glycol (PEG) was mixed with cell mass obtained from submerged fermentation to modify water activity (a_{w}) and different sugars and polyols were added as protective substances. The end product is a liquid, which is stored as such, and which does not include any solid carrier material.

Wall and Prasad in U.S. Pat. No. 5,587,158 claim a preparation of Chondrostereum purpureum made by solid state fermentation on a carrier containing powdered talc and kaolin. The colonized growth medium is refrigerated and stored aseptically as such. A formulation is made upon application on wood stumps by mixing the medium with dilute sucrose solution (less than 5% sucrose), vegetable oil, egg yolk and powdered cellulose. The end product described in this patent is essentially a wettable powder. The paste is made for application purposes, not to stabilize the microorganism in the product for storage.

The WO0182704 discloses sprayable formulations made by solid state fermentation. Microbes are cultivated on particulate carrier, such as fine peat, and stored in this form. The solid medium is suspended in water containing an optional thickening agent just prior to its application by spraying. In this method the product is stored in a dry state, not suspension. Products obtained using this method are wettable powders, which have to be suspended in liquid upon application to enable spraying.

Blachere et al. (Ann. Zool. Ecol. Anim. 5, 69-79, 1973) cultivated Beauveria brongniartii by submerged liquid fermentation and harvested the cell mass by centrifugation before mixing with silica powder, osmotically active materials (such as sucrose and sodium glutamate), anti-oxidizing agents (sodium ascorbate) and a mixture of liquid paraffin-polyoxyethylene glycerin oleate. The resultant was then dried at 4° C. in ventilated drying closet. Blastospores dried in this fashion were viable for 8 months at 4° C. This method describes a conventional liquid fermentation process followed by cell separation and drying. The formulation step is made in order to improve the stability of the product in drying. There is no suggestion that the product could be stored as a paste.

### BRIEF SUMMARY OF THE INVENTION

The object of the present invention is to provide a stable storage paste of microbial inoculants, which is easy to apply and which can be stored for long period of time without substantial deterioration. The shelf life of the products should be at least 2 months, preferably 6 months, and most preferably 12 months.

Another object is to provide a simple method for the production of a stable storage paste of inoculants containing living microorganisms.

It was surprisingly found that when growth media containing solid carrier with microorganisms grown thereon were mixed with solutions containing various protective substances, paste-like viscous suspensions were obtained having excellent long-term stability of the viable units.

Thus the inoculants can be produced without having to separate the cell mass or the spores from the growth medium and without having to dry the microbial cells or spores. Microorganisms, which do not form spores and thus cannot be dried at all can easily be stabilized according to this invention.

### DETAILED DESCRIPTION OF THE INVENTION

SSF is commonly used for the production of microbial inoculants, biological control agents in particular, since it is an efficient way of obtaining high densities of durable spores. If the carrier is correctly chosen it is not necessary to separate the cells or spores from the growth medium which makes the down stream processing extremely simple compared to submerged cultivation. Such sophisticated carriers have been described in WO9218623, the whole contents of which are included here by reference. New technologies have recently been developed to fully utilize the advantages of SSF and to make microbial inoculants better applicable. Such technologies have been described in the nonpublic patent application FI20041253, the whole contents of which is included here by reference.

Various types of reactors for growing microbes on solid culture media have been developed for solid state fermentations as shown by Mitchell et al., Process Bio-chemistry 35 (2000) 1211-1225. These include packed bed reactors, rotating drum reactors, gas-solid fluidized bed reactors and reactors wherein mixers of different kind (see US-patent publication 2002031822) have been used.

According to the method of the invention a solid carrier is used, which comprises one or more organic or inorganic carriers or both. The inorganic carriers are preferably such as kaolin, bentonite, talc, gypsum, chitosan, vermiculite, perlite, amorphous silica or granular clay or a mixture thereof. These types of materials are commonly used because they form loose, airy granular structure having preferably a particle size of 0.5-50 mm and a high surface area. The organic carriers are preferably such as cellulose, cereal grains, bran, sawdust, peat or wood chips or a mixture thereof.

A preferred solid carrier is amorphous silica, which can absorb moisture more than two times of its own weight. The granular, airy and loose structure of the moist silica medium is excellent for solid cultivations. Other inert, small particle size carrier powders such as kaolin, bentonite, talc, gypsum, chitosan or cellulose can also be added to the medium together with silica.

In addition, the solid growth medium may contain supplemental nutrients for the microorganism. Typically, these include carbon sources such as carbohydrates (sugars, starch), proteins or fats, nitrogen sources in organic form (proteins, amino acids) or inorganic nitrogen salts (ammonium and nitrate salts, urea), trace elements or other growth factors (vitamins, pH regulators). The solid growth medium may contain aids for structural composition, such as super absorbents, for example polyacrylamides. The solid carrier can also contain ingredients, which improve the applicability of the final formulation, such as oils, emulsifiers and dispersants.

The micro-organisms to be cultivated for the inoculants comprise fungi, including yeasts, for example such as Phlebiopsis gigantea, Gliocladium sp., Nectria pityrodes, Chondrostereum purpureum, Pseudozyma flocculosa, Coniothyrium minitans, Trichoderma sp., Metarrhizium sp., Verticillium sp., Myrothecium sp. or Beauveria bassiana. Preferably the fungi are Phlebiopsis gigantea, Gliocladium catenulatum, Nectria pityrodes, Myrothecium sp. or Chondrostereum purpureum. The fungi additionally include edible mushrooms such as Agaricus bisporus, Lentinus edodes or Pleurotus ostreatus. The microorganism according to the invention can be bacteria such as Streptomyces sp., Bacillus thuringiensis, other Bacillus sp. or Pseudomonas sp., preferably Streptomyces sp. In addition, nematodes could be used as microorganism to be cultivated according to the invention.

The inoculum is fed to the growth medium in liquid or solid form.

If liquid media is used as inoculum it can be in the form of for example suspension with a small particle size to enable the use of spraying techniques.

If the inoculum is in solid form it can be transported to the point of inoculation similarly to transporting the solid growth medium. Preferably, the solid inoculum is transported using a screw, vibrator or belt conveyor. This ensures that the microorganism can be transported equally aseptically for cultivation.

Incubation of the microbe on the solid growth medium usually takes 1-5 weeks depending on the cultivation conditions, nutrients and the microbe itself. Spores are in most cases the preferred form of living unit when sporulating microbes are cultivated.

When the growth medium is colonized by the microorganism, the growth medium with the microorganisms is mixed with a solution containing one or more protective substances, functioning as for example an osmotic agent. The protective substance may be selected from osmotically active substances, sugars, polyols or polymers like sucrose, fructose, lactose, trehalose, glycerol, sorbitol, glycinebetaine, polyacrylamide, polyethylene glycol, polypropylene glycol, carboxymethyl cellulose, starch and pectin or mixtures thereof. Preferably the protective substance is selected from sucrose, lactose, trehalose, sorbitol, glycinebetaine, polyacrylamide. The mixture is stirred to obtain a homogenous, viscous, paste-like suspension. The viscous paste-like suspension may be from a pourable suspension-like paste to a solid-like paste depending on the water content.

In the present invention when forming the paste-like suspension water is used in such an amount that the water contents of the paste is over 35 weight-%. When enough water is used the intensively growing filaments bind the growth substrate and a large solid cluster may be formed. For example in U.S. Pat. No. 5,587,158 the water contents is kept low (less than 25%) in order to suppress excessive growth, which would lead to an unwanted solid cluster. In the present invention clusters are wanted and subsequently crushed into fine particles of less than 150 μm with homogenisation when the paste formulation is made. This way the final paste is of uniform quality and a solution may be formed, which does not block the nozzles of the spraying equipment.

The product of the present invention is an inoculant in a form of a stable storage paste comprising 0.25-5 weight-% of a microorganism, 5-25 weight-% of a solid carrier, 5-35 weight-% of a protective substance and up to 100 weight-% of water. Preferably the inoculant comprises 0.5-1 weight-% of a microorganism, 10-15 weight-% of a solid carrier, 5-15 weight-% of a protective substance and up to 100 weight-% of water.

The pH of the product can easily be adjusted with common acids (e.g. phosphoric acid), bases or buffers (e.g. phosphate buffers). Preferred pH of the product is under about 4.

The paste-like suspensions are packed into closed packages of suitable size and stored, preferably cooled at +4-+8° C., frozen or in room temperature for short periods. The stored inoculant paste consists of 35 to 90 weight-% of water, preferably about 70 weight-% water.

When a working solution is prepared from the storage paste for applying, the paste is mixed with water to form a homogeneous solution. No special mixing equipment or additional substances are needed, and thus the applying is easy regardless of the circumstances.

The present invention is further described by the following non-limiting examples.

### EXAMPLE 1

Phlebiopsis gigantea (Rotstop, trademark of Verdera Oy) was cultivated on a silica based solid growth medium.

Nutrient solution suitable for P. gigantea was prepared by dissolving 9 g of condensed distiller's grain (CDG, Altia Oyj) to 33 g of tap water. The solution was mixed in a beaker with 15 g of amorphous silica powder (Degussa) to form a granular growth medium. 700 mg of lime was added prior to mixing to control the pH. The medium was sterilized in an autoclave for 30 min at 121° C.

The cooled medium was inoculated with 1 ml of spore suspension obtained by suspending P. gigantea spores from a potato-dextrose agar dish to sterile water. The fungus was cultivated at 28° C. for 10 days until colonized and sporulated throughout the whole medium.

10 g of the colonized medium was mixed with 10 g of a solution containing 2.5 g of protectants and 7.5 g of sterile water to form a viscous paste-like suspension having a water content of about 70%. The protective substances were
1) trehalose
2) sorbitol
3) trehalose/sorbitol (50/50)
4) trehalose/glycinebetaine (50/50)

The paste was homogenized prior to storage with Ultra Turrax homogenizer to form an even small particle size suspension of less than 150 μm.

The suspensions were placed into closed plastic sample holders, which were stored at +4° C. in a refrigerator. The viabilities of the suspensions were determined monthly:

TABLE 1
Storage stability of P. gigantea in suspension formulations.

| | |
|---|---|
| Storage time, | Viable units, cfu/g |

| months | 1 | 2 | 3 | 4 |
|---|---|---|---|---|
| 0 | 4 * 10⁷ | 4 * 10⁷ | 4 * 10⁷ | 4 * 10⁷ |
| 1 | 5 * 10⁷ | 4 * 10⁷ | 4 * 10⁷ | 3 * 10⁷ |
| 2 | 6 * 10⁷ | 3 * 10⁷ | 3 * 10⁷ | 2 * 10⁷ |
| 4 | 4 * 10⁷ | 3 * 10⁷ | 2 * 10⁷ | 2 * 10⁷ |
| 5 | 4 * 10⁷ | 1 * 10⁷ | 2 * 10⁷ | 2 * 10⁷ |

The results indicate that P. gigantea remained viable in the suspension formulations at least for 5 months.

The paste was used for stump treatment by a forest harvester against a severe pathogenic fungus Heterobasidion annosum. A working solution was made by mixing 25 g of paste to 25 liters of water. The solution was sprayed on spruce stumps through standard stump treatment equipment. The application was similar to other stump treatment agents.

### EXAMPLE 2

Chondrostereum purpureum—fungus was cultivated on a medium containing 0.8 g soluble 16-9-22 garden fertilizer (Kemira GrowHow Oyj), 15 g malt syrup (Oy Maltax AB), 359 g water and 150 g amorphous silica powder (Degussa). The medium was mixed and autoclaved as in example 1. The fungus was cultivated 11 days at 22° C. until the growth medium was completely colonized.

10 g of the colonized medium was mixed with 10 g of a solution containing 2.5 g of protectant and 7.5 g of sterile water to form a viscous paste-like suspension containing 72% of water. The protective substances were
1) trehalose
2) sorbitol
3) trehalose/sorbitol (50/50)
4) sucrose

The samples were stored and the viabilities were analyzed as in example 1.

TABLE 2
Storage stability of C. purpureum in suspension formulations.

| | |
|---|---|
| Storage time, | Viable units, cfu/g |

| months | 1 | 2 | 3 | 4 |
|---|---|---|---|---|
| 0 | 6 * 10⁵ | 6 * 10⁵ | 6 * 10⁵ | 2 * 10⁶ |
| 1 | 6 * 10⁵ | 3 * 10⁵ | 9 * 10⁵ | 1 * 10⁶ |
| 2 | 5 * 10⁵ | 7 * 10⁵ | 6 * 10⁵ | — |
| 3 | 4 * 10⁵ | 7 * 10⁵ | 8 * 10⁵ | — |
| 5 | 9 * 10⁵ | 9 * 10⁵ | 1 * 10⁶ | — |
| 6 | 9 * 10⁵ | 5 * 10⁵ | 1 * 10⁶ | — |
| 8 | 9 * 10⁵ | 8 * 10⁵ | 1 * 10⁶ | — |
| 9 | 6 * 10⁵ | 9 * 10⁵ | 2 * 10⁶ | — |
| 12 | 9 * 10⁵ | 3 * 10⁵ | 3 * 10⁶ | |

The results showed that C. purpureum had an excellent stability in suspension formulations at least for 12 months.

C. purpureum paste was homogenised prior to storage as described in example 1. The paste was used for sprout forest control by making a 1:10 dilution and by treating the sprout stumps with a brush.

### EXAMPLE 3

Fungus Myrothecium sp. was cultivated on a medium containing 3.0 g condensed distiller's grain, 34.5 g water, 0.6 g lime and 15 g amorphous silica powder (Degussa). The medium was mixed and autoclaved as in example 1. The fungus was cultivated 15 days at 18° C. until colonized and sporulated throughout the whole growth medium.

10 g of the colonized medium was mixed with 10 g of a solution containing 2.5 g of protectant and 7.5 g of 0.5% polyacrylamide solution in sterile water to form a viscous paste-like suspension containing 71% of water. The protective substances were
1) trehalose
2) sorbitol
3) trehalose/glycinebetaine (50/50)

The samples were stored and the viabilities were analyzed as in examples 1 and 2.

Myrothecium sp. paste was homogenized prior to storage with Ultra Turrax homogenizer to form an even small particle size suspension.

TABLE 3
Storage stability of Myrothecium sp. in suspension formulations.

| Storage time, | Viable units, cfu/g | | |
|---|---|---|---|
| months | 1 | 2 | 3 |
| 0 | 4 * 10⁷ | 4 * 10⁷ | 4 * 10⁷ |
| 1 | 4 * 10⁷ | 5 * 10⁷ | 5 * 10⁷ |
| 3 | 2 * 10⁷ | 5 * 10⁷ | 1 * 10⁷ |

Myrothecium sp. was viable in the suspension formulations for at least 3 months.

The paste was used as such for coating of grass seeds with standard seed coating equipment. Myrothecium sp. acts as a germination and growth stimulator for the seeds.

### EXAMPLE 4

Streptomyces sp. strain K61 bacterium (Mycostop, trademark of Verdera Oy) was cultivated on a solid growth medium containing 5.2 g corn steep solids (CSS, Roquette, France), 5.2 lactose (Merck) 5.2 g lime, 100 g amorphous silica powder (Degussa) and 240 g tap water. The medium was mixed and autoclaved as in example 1. The bacterium was cultivated 7 days at 28° C.

10 g of the colonized medium was mixed with 10 g of
1) 10% sucrose solution (78% water in the product)
2) 20% sucrose solution (73% water in the product)

The samples were stored in plastic sample holders in a refrigerator at +4° C.

TABLE 4
Storage stability of Streptomyces sp. in suspension formulations.

| | |
|---|---|
| Storage time, | Viable units, cfu/g |

| months | 1 | 2 |
|---|---|---|
| 0 | 2 * 10⁹ | 2 * 10⁹ |
| 1 | 2 * 10⁹ | 7 * 10⁸ |
| 2 | 1 * 10⁹ | 1 * 10⁹ |
| 3 | 8 * 10⁸ | 8 * 10⁸ |
| 4 | 1 * 10⁹ | 1 * 10⁹ |
| 5 | 1 * 10⁹ | 1 * 10⁹ |
| 6 | 1 * 10⁹ | 1 * 10⁹ |
| 12 | 1 * 10⁹ | 1 * 10⁹ |

The results showed that Streptomyces sp. had an excellent stability in suspension formulations at least for 12 months.

### EXAMPLE 5

Myrothecium sp. fungus was cultivated on four different media:

| Ingredient | 1 | 2 | 3 | 4 |
|---|---|---|---|---|
| Nutrient solution: | | | | |
| 1) 7.8% CDG-solution | 37.5 | 34.5 | 36.0 | |
| 2) solution from Ex 2 | | | | 37.5 |
| lime | 0.6 | 0.6 | 0.6 | |
| amorphous silica | 15 | 13.5 | 13.5 | 15 |
| kaolin* | | 1.5 | | |
| cellulose powder** | | | 1.5 | |

(*ECC International, **Penwest Pharmaceuticals)

The media were mixed and autoclaved as in example 1. The fungus was cultivated 15 days at 18° C. except on medium 4, which was cultivated for 3 months until the media were completely colonized.

10 g each of the colonized medium was mixed with 10 g of solution containing 2 g sucrose and 8 g 0.5% polyacrylamide solution to form suspensions containing about 74% of water. The samples were stored as in example 1.

TABLE 5
Storage stability of Myrothecium sp. in suspension formulations.

| | |
|---|---|
| Storage time, | Viable units, cfu/g |

| months | 1 | 2 | 3 | 4 |
|---|---|---|---|---|
| 0 | 1 * 10⁷ | 1 * 10⁷ | 1 * 10⁷ | 3 * 10⁷ |
| 1 | 5 * 10⁶ | 4 * 10⁶ | 6 * 10⁶ | 2 * 10⁷ |
| 3 | 4 * 10⁶ | 4 * 10⁶ | 2 * 10⁶ | 1 * 10⁷ |
| 4 | 3 * 10⁶ | 3 * 10⁶ | 3 * 10⁶ | 1 * 10⁷ |
| 7 | 3 * 10⁶ | 1 * 10⁶ | 1 * 10⁶ | 1 * 10⁷ |

The results indicated that Myrothecium sp. survived in suspesion formulations at least for 7 months.

### EXAMPLE 6

Gliocladium catenulatum fungus (Prestop, trademark of Verdera Oy) was cultivated on a medium containing 5.3 g condensed distiller's grain, 33.8 g water, 0.53 g lime and 15 g amorphous silica powder (Degussa). The medium was mixed and autoclaved as in example 1. The fungus was cultivated 15 days at 18° C. until colonized and sporulated throughout the whole growth medium.

10 g of the colonized medium was mixed with 10 g of
1) 10% sucrose solution (79% water in the product)
2) 20% sucrose solution (74% water in the product)
3) 10% lactose solution (79% water in the product)
2) 20% lactose solution (74% water in the product)

The samples were stored and the viabilities were analyzed as in example 1.

TABLE 6
Storage stability of G. catenulatum in suspension formulations.

| | |
|---|---|
| Storage time, | Viable units, cfu/g |

| months | 1 | 2 | 3 | 4 |
|---|---|---|---|---|
| 0 | 5 * 10⁷ | 5 * 10⁷ | 5 * 10⁷ | 5 * 10⁷ |
| 1 | 3 * 10⁷ | 3 * 10⁷ | 3 * 10⁷ | 3 * 10⁷ |
| 3 | 2 * 10⁷ | 2 * 10⁷ | 2 * 10⁷ | 2 * 10⁷ |
| 5 | 3 * 10⁷ | 2 * 10⁷ | 2 * 10⁷ | 3 * 10⁷ |
| 8 | 3 * 10⁷ | 7 * 10⁷ | 2 * 10⁷ | 4 * 10⁷ |
| 10 | 2 * 10⁷ | 3 * 10⁷ | — | — |
| 12 | 1 * 10⁷ | 2 * 10⁷ | — | — |

The results showed that G. catenulatum had an excellent stability in suspension formulations at least for 12 months.

About 60 kg of formulation 2 was homogenised with a 100 liter dispergator prior to storage. The paste was applied by spraying to turf grass on a golf course for controlling a common disease, snow mould. A working solution was made by mixing 10 kg of paste with 500 to 1000 liters of water, and the turf was treated by a standard sprayer.

It is understood that the disclosed invention is not limited to the particular methodology, protocols, and reagents described as these may vary. It is also to be understood that the terminology used herein is for the purpose of describing particular embodiments only, and is not intended to limit the scope of the present invention, which will be limited only by the appended claims.

1. An inoculant in a form of a stable storage paste comprising (% w/w):
a) 0.25-5% microorganism
b) 5-25% solid carrier
c) 5-35% protective substance
d) up to 100% water.

2. The inoculant of claim 1 comprising (% w/w):
a) 0.5-2% microorganism
b) 10-20% solid carrier
c) 5-15% protective substance
d) up to 100% water.

3. The inoculant of claim 1, wherein the microorganism is a fungus, yeasts, bacterium, or nematode.

4. The inoculant of claim 3, wherein the microorganism is Phlebiopsis gigantea, Chondrostereum purpureum, Gliocladium catenulatum, Nectria pityrodes, Myrothecium sp., or Streptomyces sp.

5. The inoculant according to claim 1, wherein the solid carrier is kaolin, bentonite, talc, gypsum, chitosan, cellulose, cereal grains, bran, sawdust, peat or wood chips, vermiculite, perlite, amorphous silica, granular clay, or a mixture thereof.

6. The inoculant of claim 5, wherein the solid carrier comprises amorphous silica.

7. The inoculant of claim 5, wherein the solid carrier comprises a mixture of
an amorphous silica and
kaolin, bentonite, talc, gypsum, chitosan, or cellulose.

8. The inoculant of claim 1, wherein the protective substance is selected from the group consisting of osmotically active substances, sugars, polyols, and polymers.

9. The inoculant of claim 8, wherein the protective substance is selected from the group consisting of sucrose, fructose, lactose, trehalose, glycerol, sorbitol, glycinebetaine, polyacrylamide, polyethylene glycol, polypropylene glycol, carboxymethyl cellulose, starch, pectin, and mixtures thereof.

10. A method for production of an inoculant in a form of a stable storage paste, comprising
a. cultivating microorganisms on a solid carrier,
b. mixing the solid carrier from step (a) containing living microorganisms and/or spores of the microorganism with a solution containing one or more protective substances to form a mixture, and
c. homogenizing the mixture to form a storage paste containing more than 35 w-% water.

11. The method according to claim 10, wherein the solid carrier is kaolin, bentonite, talc, gypsum, chitosan, cellulose, cereal grains, bran, sawdust, peat or wood chips, vermiculite, perlite, amorphous silica, granular clay, or a mixture thereof.

12. The method of claim 11, wherein the solid carrier comprises amorphous silica.

13. The method of claim 12, wherein the solid carrier comprises a mixture of
an amorphous silica and
kaolin, bentonite, talc, gypsum, chitosan, or cellulose.

14. The method of claim 10, wherein the microorganism is a fungus, yeasts, bacterium, or nematode.

15. The method of claim 14, wherein the microorganism is Phlebiopsis gigantea, Chondrostereum purpureum, Gliocladium catenulatum, Nectria pityrodes, Myrothecium sp., or Streptomyces sp.

16. The method of claim 10, characterized in that wherein the protective substance is selected from the group consisting of osmotically active substances, sugars, polyols, and polymers.

17. The method of claim 16, wherein the protective substance is selected from the group consisting of sucrose, fructose, lactose, trehalose, glycerol, sorbitol, glycinebetaine, polyacrylamide, polyethylene glycol, polypropylene glycol, carboxymethyl cellulose, starch, pectin, and mixtures thereof.
